(19) United States
(12) Patent Application Publication
(10) Pub. No.: US 2018/0185769 A1
(43) Pub. Date: Jul. 5, 2018

(54) Membrane device and method for manufacturing the membrane device

(71) Applicant: Janesko Oy, Vantaa (FI)

(72) Inventor: Liangyu CHEN, Vantaa (FI)

(73) Assignee: Janesko Oy, Vantaa (FI)

(21) Appl. No.: 15/849,777

(22) Filed: Dec. 21, 2017

(30) Foreign Application Priority Data
Dec. 29, 2016 (FI) 20166042

(51) Int. Cl. B01D 19/00; B01D 69/02; B01D 69/12; B01D 71/70; B01D 71/36; B01D 71/26; G01N 33/00

(52) U.S. Cl. CPC B01D 19/0031; B01D 69/02; B01D 69/12; B01D 2325/38; B01D 71/36; B01D 71/26; G01N 33/0011; B01D 71/70

(57) ABSTRACT

A membrane device for defining interface between first and second environments and for selectively passing through at least one component of a solution of at least two different components from the first environment to the second environment comprises at least one first film layer structure, and at least one second film layer structure. The first layer structure comprises first hydrophobic material, the contact angle of which between the first hydrophobic material and water is more than 90°. In addition, the second film layer structure comprises second hydrophobic material, the contact angle of said second hydrophobic material and water being not greater and advantageously less than said contact angle between said first hydrophobic material and water. The membrane device comprises advantageously also a support structure for supporting at least one of said film layer structure. The support structure is e.g. a polypropylene or stainless-steel mesh, and the support structure as a whole is porous structure.

### PRIORITY

This application claims priority of Finnish patent application number FI20166042 filed on Dec. 29, 2016, the contents of which is incorporated herein by reference.

### TECHNICAL FIELD OF THE INVENTION

The invention relates to a membrane device and method for manufacturing the membrane device. Especially the invention relates to the membrane device for defining interface between first and second environments and for selectively passing or separating through at least one component of a solution of at least two different components from the first environment to the second environment.

### BACKGROUND OF THE INVENTION

There is a need for determining concentration of gaseous component from a liquid solution having at least two different components. One method for determining is to lead gaseous component from the liquid solution through a membrane into a measuring chamber and into a contact of a suitable detector. There are few challenges in the method, namely at first the desired gaseous component should be selectively separated from the solution so that non-desired components are not passed. Additionally the measuring chamber is typically a dry measuring environment, whereupon the interface between the liquid solution and the measuring environment must be essentially impermeable to liquids.

Few methods are known from prior art, such as using a composite membrane having a porous support layer and a non-porous permselective membrane layer. It is also known to use a membrane for sensors that detects gases in oil-filled devices, where on the feed side a gas permeable polymer membrane is used and on the permeate side a porous sintered metal is used. Layers are connected with gas permeable adhesive. In many known applications the pressure on the feed side is normal air pressure and the pressure in the permeate side is close to vacuum. This means the maximum pressure difference is maximum 100 kPa.

There are however some drawback related to the known prior art solutions, namely one problem relates to environments with high pressure difference between the environments. For example in certain situations the pressure difference might be around 500 kPa or even more than 1000 kPa, which raises demands for a reliable way to separate the desired gaseous component without any solution leakage. In addition there is a problem with the prior art solutions of wetting of a membrane in high pressure environments when filtering gas from liquid solvents (especially water). In such environment solvent penetrates the membrane and deteriorate the permeation properties of the membrane. Penetrated solvent might affect the sensor operation or even destroy it.

### SUMMARY OF THE INVENTION

An object of the invention is to alleviate and eliminate the problems relating to the known prior art. Especially the object of the invention is to provide a membrane structure for efficiently and in a reliable way to selectively passing and separating at least one desired component of a solution of at least two different components from a first environment having the solution with the components to a second environment being connected to a measuring detectors.

The object of the invention can be achieved by the features of independent claims.

The invention relates to a membrane device according to claim 1. In addition the invention relates to a method for manufacturing the membrane device according to claim 15.

According to an embodiment of the invention a membrane device for defining interface between first and second environments and for selectively passing and separating through at least one component of a solution of at least two different components from a first environment to a second environment is provided. The solution, advantageously liquid solution is kept in the first environment and a measuring chamber is located in or connected to the second environment.

The membrane device advantageously comprises at least one first film layer structure and additionally also at least one second film layer structure coupled with each other. The first layer structure to be faced to the first environment comprises first hydrophobic material, which is selected so that the contact angle between said first hydrophobic material and water is more than 90° so to prevent solvent (water) from permeating to the next layers. The second film layer structure comprises second hydrophobic material, which is selected so that the contact angle between said second hydrophobic material and water is not greater and advantageously less than said contact angle between said first hydrophobic material and water.

Advantageously the second material is not so hydrophobic as the first one and the layers are arranged in such a way that the layer with the most hydrophobic material is located on the feed side of the membrane (faced to the first environment) and other layers are arranged advantageously in descending hydrophobicity. By this the problems relating to the wetting of the membrane in high pressure environments e.g. in filtering gas from liquid solvents (especially water) can be avoided or at least minimized.

According to an exemplary embodiment the first hydrophobic material is silicone (PDSM) or polytetrafluoroethylene (PTFE/Teflon), and the second hydrophobic material is polytetrafluoroethylene (PTFE/Teflon) or polypropylene (PP). Advantageously the second film layer structure is a permselective layer, which essentially allows only first desired gaseous component to permeate through the membrane device. The desired permselectivity can be determined for example by size of pores of the material, but also selection of diffusion and/or absorption coefficient of the material can be used for desired outcome.

According to an example the layers of the membrane device are chosen so to selectively passing through at least one gaseous component of the solution, wherein said gaseous component is for example CO₂, O₂ or ethanol, however not limiting to those only. According to an exemplary embodiment the first film layer structure can be made for example by dipping a bulk selective film layer structure to a silicone rubber solution, which can then be stacked with a supportive layer (described elsewhere in this document). Naturally, also other methods can be applied.

In addition, according to an advantageously embodiment the membrane device comprises also a support structure for supporting at least one of said film layer structure, thereby strengthening the mechanical structure of the membrane device resisting the pressure difference between the feed side (faced towards the first environment) and the permeate side (faced towards the second environment) of the membrane device. The support structure comprises for example a polypropylene or stainless steel mesh, and it is as a whole porous structure, so advantageously more porous than the first and/or second film layer structures, thereby minimizing any effect of it to the separation and selection of the component(s) of the solution through the membrane device.

The support structure can be coupled to the membrane device in many ways, such as integrated into one of the hydrophobic (first) layers or between one or more film layer structures, or integrated into a contact of the second side (permeate side) of the second film layer structure, depending on the application. It has been noticed by the inventor that integrating the support to one of the hydrophobic or selective layers is very advantageously namely then the risk of layers separating from each other due to (possible) weak bonding of them can be minimized. In addition changing pressure may easily cause movement in the membrane device that will cause stress to the bonding, for example.

The support structure is advantageously configured to support said film layer structures so to persist at least the pressure difference of at least 500 kPa, more advantageously at least 750 kPa and most advantageously more than 1000 kPa between the first and second environments. This can be achieved e.g. by using the polypropylene or stainless steel mesh, which is coupled or integrated to the membrane device structure as described elsewhere in this document.

The present invention offers advantages over the known prior art, such as already discussed elsewhere in this document, and especially allowing selective passing and separating at least one component of a solution of at least two different components though the membrane device from the first environment to the second environment. In addition the invention offers a solution to prevent the wetting of the membrane in high pressure environments, for example when filtering gas from liquid solvents (especially water).

The exemplary embodiments presented in this text are not to be interpreted to pose limitations to the applicability of the appended claims. The verb “to comprise” is used in this text as an open limitation that does not exclude the existence of also unrecited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objects and advantages thereof, will be best understood from the following description of specific example embodiments when read in connection with the accompanying drawings.

### BRIEF DESCRIPTION OF THE DRAWINGS

Next the invention will be described in greater detail with reference to exemplary embodiments in accordance with the accompanying drawings, in which:

FIG. 1 illustrates a principle of an exemplary membrane device for defining interface between first and second environments according to an advantageous embodiment of the invention,

FIGS. 2A-2B illustrate exemplary implementations of the membrane device with a supporting structure according to an advantageous embodiment of the invention, and

FIG. 3 illustrates an exemplary arrangement, where the membrane device according to an advantageous embodiment of the invention can be used.

### DETAILED DESCRIPTION

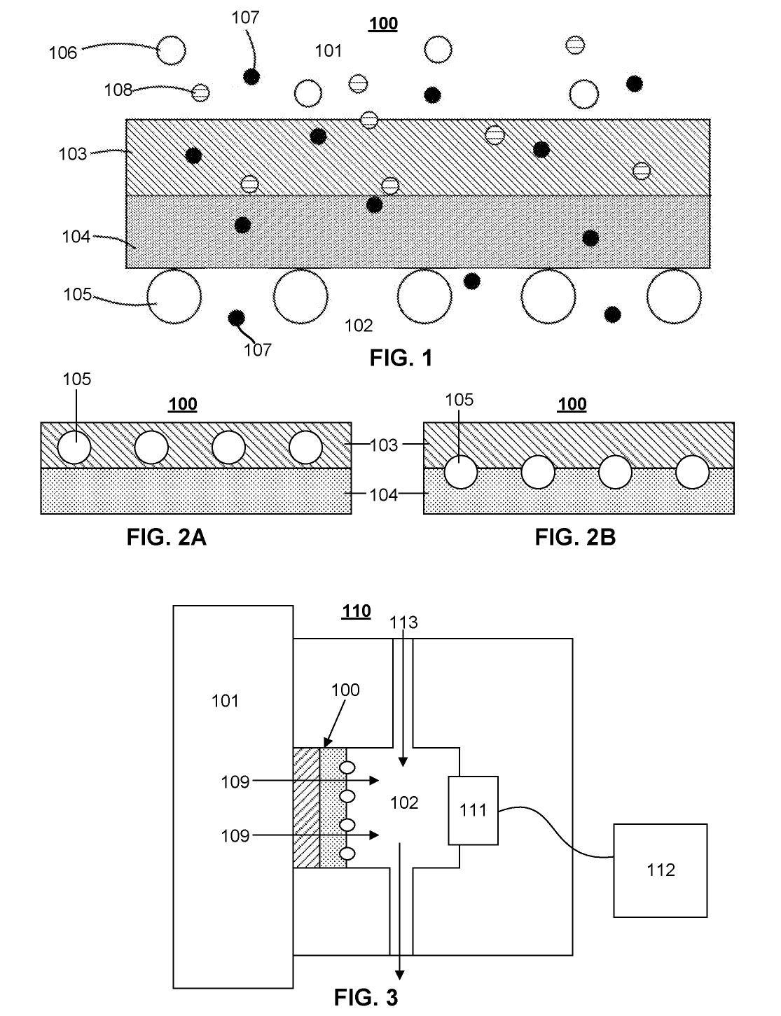

FIG. 1 illustrates a principle of an exemplary membrane device 100 for defining interface between first 101 and second 102 environments according to an advantageous embodiment of the invention, and used to separate solution and dissolved gas from each other with high pressure difference between two sides of the membrane for gas analyzation that is based on absorption

The membrane device 100 comprises the first film layer structure 103 in the “feed side” to be faced against the first environment 101 and the second film layer structure 104 next to the first film layer structure and towards the “permeate side” and the second environment 102. In addition the membrane device comprises a supportive structure 105 in addition to said one or more permselective and hydrophobic layers 103, 104.

The hydrophobic layer 104 as the first film layer structure on the feed side prevents solvent (typically water 106 from permeating to the next layers. Inner permselective layer(s) 104 is/are as the second film layer structure. This means that its/their 104 main function is to permeate wanted gases 107 or gaseous substances and prevents unwanted gas 108 or gaseous substances from permeating the membrane device 100. The pressure difference across the membrane can be over 1000 kPa, from what the supportive layer 105 is opposing.

The supportive layer 105 on the permeate side is advantageously porous mechanical support, such as polypropylene (PP) or stainless steel mesh. Other layers 103, 104 are hydrophobic membranes, such as silicone (PDMS) of polytetrafluoroethylene (PTFE), organized such way that the most hydrophobic is on the feed side and other layers are organized in descending hydrophobicity, advantageously. The possible middle layers (not shown) can be permselective to only allow desired gaseous molecules to permeate.

However, it is to be noted that the supportive layer 105 as the support structure is optional feature, even if it shown in Figures.

FIGS. 2A-2B illustrate exemplary implementations of the membrane device 100 with the supporting structure 105 according to an advantageous embodiment of the invention. In FIG. 2A embodiment the support structure is provided into the first film layer structure 103, whereas in FIG. 2B embodiment the support structure is provided between the first and second film layer structures 103,104. One advantageous embodiment is illustrated in FIG. 1, where the supporting structure 105 is integrated into the contact of the second film layer structure 104.

FIG. 3 illustrates an exemplary arrangement 110, where the membrane device 100 according to an advantageous embodiment of the invention can be used. The membrane device 100 defines an interface between the first 101 and second 102 environments, as is described elsewhere in this document. The first and second film layer structures 103, 104 are selected so that at least one desired component of the solution can be separated and passed 109 selectively from the first environment 101 to the second environment 102, so to the dry measuring chamber 102 in this embodiment.

Cleaning gas is provided 113 so to removing the components derived through 109 the membrane device and keeping the measuring chamber 102 dry at the same time. The detector 111 and suitable data processing unit 112 can then be used for determining e.g. concentration of the components passed the membrane device.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims.

In particularly it is to be noted that even if two film layer structures 103, 104 are described, the amount of layers can be varied and in the embodiment described it is reduced to only two by selecting the layers that is/are both very hydrophobic and also permselective to the wanted gaseous substance.

The features recited in dependent claims are mutually freely combinable unless otherwise explicitly stated.

1. A membrane device for defining interface between a first and a second environment and for selectively passing through at least one component of a solution of at least two different components from the first environment to the second environment,
wherein the membrane device comprises:
at least one first film layer structure, wherein the first film layer structure comprises first hydrophobic material, wherein a contact angle between said first hydrophobic material and water is more than 90°; and
at least one second film layer structure, wherein the second film layer structure comprises second hydrophobic material, wherein a contact angle between said second hydrophobic material and water being not greater and advantageously less than said contact angle between said first hydrophobic material and water; and wherein
the membrane device comprises additionally a support structure, wherein the support structure is integrated into or between one or more film layer structures.

2. The membrane device of claim 1, wherein the first hydrophobic material is silicone (PDSM) or polytetrafluoroethylene (PTFE).

3. The membrane device of claim 1, wherein the first film layer structure is made by dipping a bulk selective film layer structure to a silicone rubber solution.

4. The membrane device of claim 1, wherein the second hydrophobic material is polytetrafluoroethylene (PTFE) or polypropylene (PP).

5. The membrane device of claim 1, wherein the first film layer structure is configured to be faced against the first environment and the second film layer structure is configured to locate after said first film layer structure in a direction from the first environment towards the second environment in use.

6. The membrane device of claim 5, wherein the second film layer structure is a permselective layer only allowing a desired gaseous first component to permeate through the membrane device.

7. The membrane device of claim 6, wherein desired permselectivity is determined by size of pores of the material, diffusion or absorption coefficient selection of the material.

8. The membrane device of claim 1, wherein the membrane device comprises also a support structure for supporting at least one of said film layer structure.

9. The membrane device of claim 1, wherein the support structure comprises a polypropylene or stainless-steel mesh, where said support structure as a whole is a porous support structure.

10. The membrane device of claim 1, wherein the support structure is configured to support said film layer structures so to persist at least a pressure difference of at least 500 kPa between the first and second environments.

11. The membrane device of claim 1, wherein the support structure is configured to support said film layer structures so to persist at least pressure difference of at least 750 kPa between the first and second environments.

12. The membrane device of claim 1, wherein the support structure is configured to support said film layer structures so to persist at least pressure difference of more than 1000 kPa between the first and second environments.

13. The membrane device of claim 1, wherein the support structure is integrated into a contact of a second side of said second film layer structure, wherein said second side of said second film layer structure is a side facing towards the second environment in use.

14. The membrane device of claim 1, wherein the membrane device is configured to selectively passing through at least one gaseous component of the solution, wherein said gaseous component is CO₂, O₂ or ethanol.

15. A method for manufacturing a membrane device, said membrane device defining interface between a first and a second environment and for selectively passing through at least one component of a solution of at least two different components from the first environment to the second environment, wherein the method comprises steps of:
providing at least one first film layer structure, wherein the first layer structure comprises first hydrophobic material, wherein a contact angle between said first hydrophobic material and water is more than 90°, and
providing at least one second film layer structure next to said at least one first film layer structure, wherein the second film layer structure comprises second hydrophobic material, a contact angle between said second hydrophobic material and water being not greater and advantageously less than said contact angle between said first hydrophobic material and water; and wherein
the method comprises additionally a step of integrating a support structure) into or between one or more film layer structures.
